(19) United States
(12) Patent Application Publication
(10) Pub. No.: US 2019/0269971 A1
(43) Pub. Date: Sep. 5, 2019

(54) CUSTOM WORKOUT SYSTEM

(71) Applicant: ICON Health & Fitness, Inc., Logan, UT (US)

(72) Inventors: Rebecca Lynn Capell, Logan, UT (US); Chase Brammer, Providence, UT (US)

(21) Appl. No.: 16/414,489

(22) Filed: May 16, 2019

Related U.S. Application Data
(63) Continuation of application No. 15/712,656, filed on Sep. 22, 2017.
(60) Provisional application No. 62/400,762, filed on Sep. 28, 2016.

(51) Int. Cl. A63B 24/00; G16H 20/30

(52) U.S. Cl. CPC A63B 24/0075; G16H 20/30; A63B 2024/0078; A63B 2024/0065; A63B 24/0062

(57) ABSTRACT

A method for customizing workout recommendations may include receiving a target workout duration for a user, determining a target calorie burn for the user, and determining the recentness of each of the workouts completed by the user. This determination may include receiving physical movement data of the user from one or more electronic sensors configured to directly measure physical movement of the user, analyzing the physical movement data, and determining whether each of the workouts was completed based on the analysis of the physical movement data. The method may further include assigning a weight to each of the workouts based on the received target workout duration, the determined target calorie burn for the user, and the determined recentness of the workout being completed by the user, ranking the workouts based on their assigned weights, and generating a custom workout recommendation for the user based on the ranking of the workouts.

### RELATED APPLICATIONS

This application is a continuation of U.S. patent application Ser. No. 15/712,656, filed Sep. 22, 2017, which claims priority to U.S. Patent Application Ser. No. 62/400,762, filed on Sep. 28, 2016. Each of these applications is herein incorporated by reference for all that it discloses.

### BACKGROUND

A workout is a bodily activity that enhances or maintains physical fitness and overall health and wellness. It is performed for various reasons, including increasing growth and development, decreasing the negative effects of aging, strengthening muscles and the cardiovascular system, honing athletic skills, weight loss or maintenance, and merely enjoyment. Frequent and regular workouts boost the immune system and help prevent diseases of affluence such as cardiovascular disease, type 2 diabetes, and obesity. Working out may also help prevent stress and depression, increase the quality of sleep, help promote or maintain positive self-esteem, and improve mental health.

Various standard and non-standard workouts have been developed by personal trainers and other exercise science practitioners. These workouts have been developed to be performed in connection with workout equipment or to be performed without the use of any workout equipment. Perhaps thousands or even tens of thousands of different workouts are available and recommended by personal trainers.

One common problem faced by individuals is selecting an appropriate workout from among the overwhelming number of choices available. One way an individual may deal with this problem is to consult with a personal trainer, who may make a recommendation based on an analysis of the individual's healthy and unhealthy habits. However, such a consultation can be expensive and time consuming, and may be unhelpful due to the individual providing inaccurate information to the personal trainer, since individuals notoriously overestimate their healthy habits and underestimate their unhealthy habits.

### SUMMARY

In one aspect of the disclosure, a method for customizing workout recommendations may include receiving a target workout duration for a user, determining a target calorie burn for the user, and determining the recentness of each of the workouts completed by the user. This determination may include receiving physical movement data of the user from one or more electronic sensors configured to directly measure physical movement of the user, analyzing the physical movement data, and determining whether each of the workouts was completed based on the analysis of the physical movement data. The method may further include assigning a weight to each of the workouts based on the received target workout duration, the determined target calorie burn for the user, and the determined recentness of the workout being completed by the user, ranking the workouts based on their assigned weights, and generating a custom workout recommendation for the user based on the ranking of the workouts.

Another aspect of the disclosure may include any combination of the above-mentioned features and may further include the one or more electronic sensors including a wearable electronic sensor configured to be worn on a wrist of the user.

Another aspect of the disclosure may include any combination of the above-mentioned features and may further include the one or more electronic sensors including an exercise machine electronic sensor.

Another aspect of the disclosure may include any combination of the above-mentioned features and may further include the method further including determining the recentness of each of the workouts being recommended but not completed by the user and the assigning of the weight to each of the workouts being further based on the determined recentness of the workout being recommended but not completed by the user.

Another aspect of the disclosure may include any combination of the above-mentioned features and may further include the method further including receiving a target muscle group for the user and the assigning of the weight to each of the workouts being further based on the received target muscle group for the user.

Another aspect of the disclosure may include any combination of the above-mentioned features and may further include the method further including determining the recentness of each of the multiple target muscle groups being a focus of the workouts completed by the user and the assigning of the weight to each of the workouts being further based on the determined recentness of a target muscle group that is a focus of the workout being a focus of the workouts completed by the user.

Another aspect of the disclosure may include any combination of the above-mentioned features and may further include the determining of the target calorie burn for the user based on the received fitness level of the user, determining a target heart rate range for the user based on the received fitness level of the user, the received mass of the user, the received sex of the user, and determining the target calorie burn for the user based on the determined target heart rate range for the user, the received mass of the user, the received sex of the user, and the received target workout duration for the user.

Another aspect of the disclosure may include any combination of the above-mentioned features and may further include the method further including receiving a target workout category goal of the user and the assigning of the weight to each of the workouts being further based on the received target workout category goal of the user.

Another aspect of the disclosure may include any combination of the above-mentioned features and may further include the method further including receiving a fitness level of the user and the assigning of the weight to each of the workouts being further based on the received fitness level of the user.

Another aspect of the disclosure may include any combination of the above-mentioned features and may further include the method further including receiving a workout equipment availability of the user and the assigning of the weight to each of the workouts being further based on the received workout equipment availability of the user.

Another aspect of the disclosure may include any combination of the above-mentioned features and may further include the method further including receiving a sex of the user and the assigning of the weight to each of the workouts being further based on the received sex of the user.

Another aspect of the disclosure may include any combination of the above-mentioned features and may further include one or more non-transitory computer-readable media storing one or more programs that are configured, when executed, to cause one or more processors to perform the method for customizing workout recommendations.

### BRIEF DESCRIPTION OF THE DRAWINGS

The accompanying drawings illustrate various embodiments of the present method and system and are a part of the specification. The illustrated embodiments are merely examples of the present system and method and do not limit the scope thereof.

FIG. 1 is a diagram of an example health system;

FIGS. 2A-2B are example webpages of an example website that may be employed in connection with the example health system of FIG. 1; and

FIGS. 3A-3B are a diagram of an example method for customizing workout recommendations.

Throughout the drawings, identical reference numbers designate similar, but not necessarily identical, elements.

### DETAILED DESCRIPTION

Methods for customizing workout recommendations are disclosed herein. Specifically, the present methods generate custom workout recommendations for users based on various data that is received or determined. For example, the received data may include a target workout duration for a user and physical movement data of the user. The received physical movement data may be received from one or more electronic sensors configured to directly measure physical movement of the user. This received physical movement data may then be analyzed and then whether each of the workouts was completed may be determined based on the analysis of the received physical movement data. Further, a target calorie burn may be determined for the user. A weight may then be assigned to each of the workouts based on the received target workout duration, the determined target calorie burn for the user, and the determined recentness of the workout being completed by the user. The workouts may then be ranked based on their assigned weights. Finally, the custom workout recommendation for the user may be generated based on the ranking of the workouts. The methods for customizing workout recommendations are described in detail below.

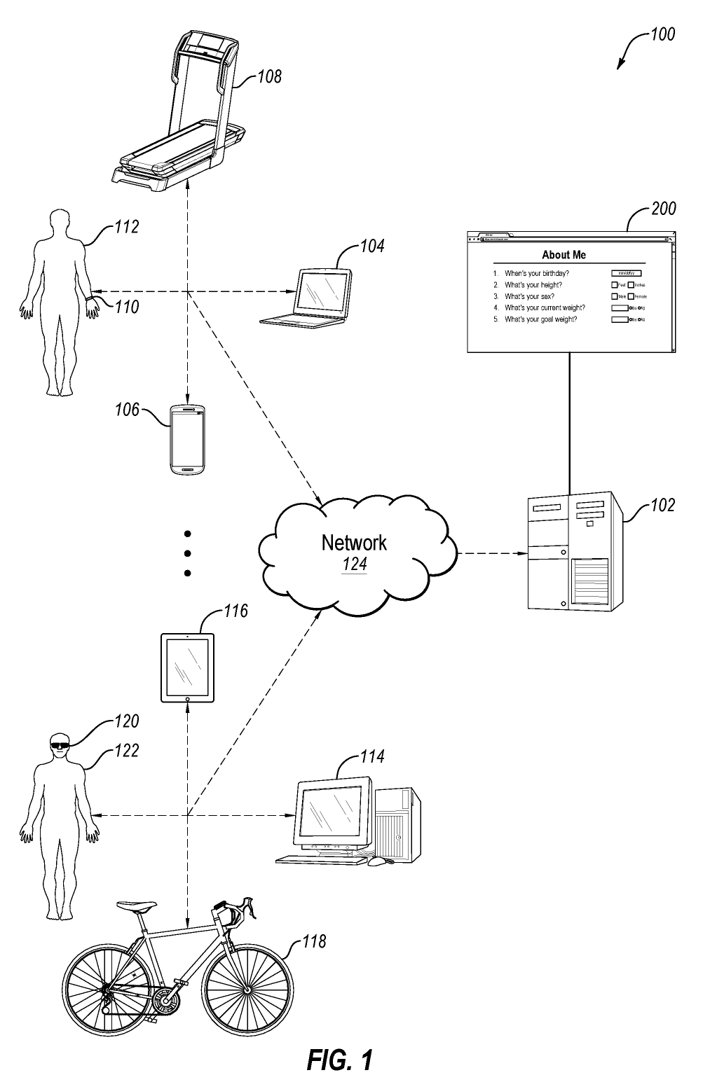

FIG. 1 is a diagram of an example health system 100. The system 100 may include a server 102 that hosts a website 200. The system 100 may also include a laptop computer 104, a smartphone 106, a treadmill 108, and an activity tracker watch 110 configured to be worn on the wrist of a first user 112. The system 100 may further include a desktop computer 114, a tablet 116, a bicycle 118, and smart glasses 120 configured to be worn by a second user 122.

As disclosed in FIG. 1, each of the computing devices in the system 100 may be configured to communicate with one another wirelessly, either locally or remotely via a network 124. In particular, the activity tracker watch 110 worn by the first user 112 may include an electronic sensor, such as an accelerometer, that is configured to directly measure physical movement of the first user 112, such as the number of steps taken by the first user 112, resulting in physical movement data. Similarly, the treadmill 108 may include multiple electronic sensors, such as an odometer, a tilt sensor, and a resistance sensor, that are configured to directly measure physical movement of the first user 112, such as the simulated distance run by the first user 112 on the treadmill 108, the incline while running, and the amount of effort expended by the first user 112 on the treadmill 108, resulting in physical movement data. The physical movement data from the activity tracker watch 110 and the treadmill 108 may be sent to, and received by, the laptop computer 104, the smartphone 106, or the server 102, or some combination thereof. A software application running on the laptop computer 104, the smartphone 106, or the server 102, or some combination thereof, may then be configured to analyze the physical movement data and then determine, based on the analysis of the physical movement data, one or more physical movement parameters. These one or more physical movement parameters may include a number of calories burned by the first user 112. After the software application has determined the one or more physical movement parameters, the software application may then generate a custom workout recommendation for the first user 112 based at least in part on the one or more physical movement parameters.

Further, the smart glasses 120 worn by the second user 122 may include multiple electronic sensors, such as a GPS receiver and a video camera, that are configured to directly measure physical movement of the second user 122, such as the distance traveled and the amount of head movement by the second user 122, resulting in physical movement data. Similarly, the bicycle 118 may include an electronic sensor, such as a cadence sensor, that is configured to directly measure physical movement of the second user 122, such as the number of pedal strokes performed by the second user 122 on the bicycle 118, resulting in physical movement data. The physical movement data from the smart glasses 120 and the bicycle 118 may be sent to, and received by, the desktop computer 114, the tablet 116, or the server 102, or some combination thereof. A software application running on the desktop computer 114, the tablet 116, or the server 102, or some combination thereof, may then be configured to analyze the physical movement data and then determine, based on the analysis of the physical movement data, one or more physical movement parameters. After the software application has determined the one or more physical movement parameters, the software application may then generate a custom workout recommendation for the second user 122 based at least in part on the one or more physical movement parameters.

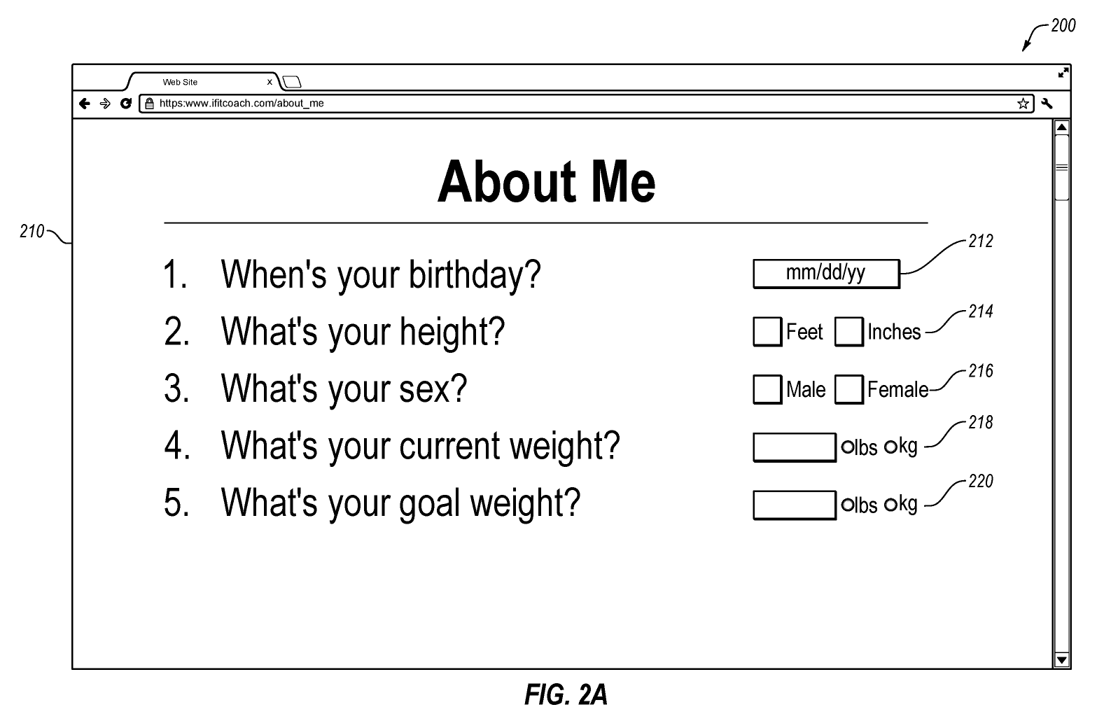

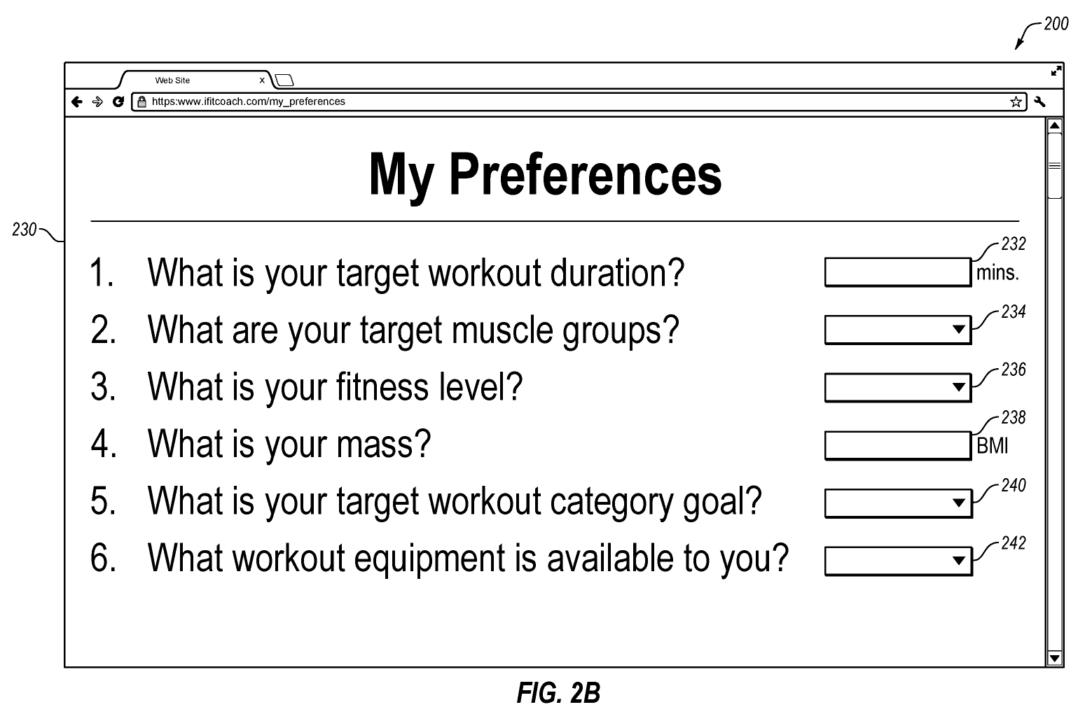

FIGS. 2A-2B are example webpages of the website 200 that may be employed in connection with the system 100 of FIG. 1.

As disclosed in FIG. 2A, a first webpage 210 of the website 200 may be configured to be presented to a user in order to receive data about the user. In particular, the first webpage 210 may be configured to receive the user's birthday, height, sex, current weight, and weight loss goal in data entry fields 212-220, respectively.

As disclosed in FIG. 2B, a second webpage 230 of the website 200 may be configured to be presented to a user in order to receive data regarding the preferences of the user. In particular, the second webpage 230 may be configured to receive the user's target workout duration, target muscle group, fitness level, mass, target workout category goal, and workout equipment availability in data entry fields 232-242, respectively.

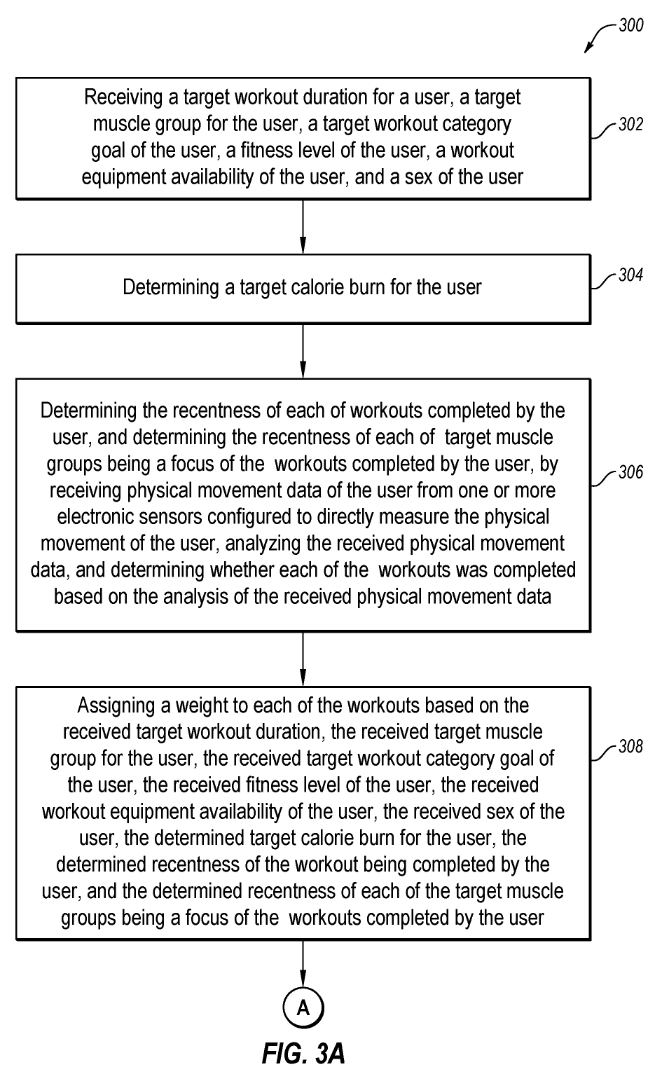

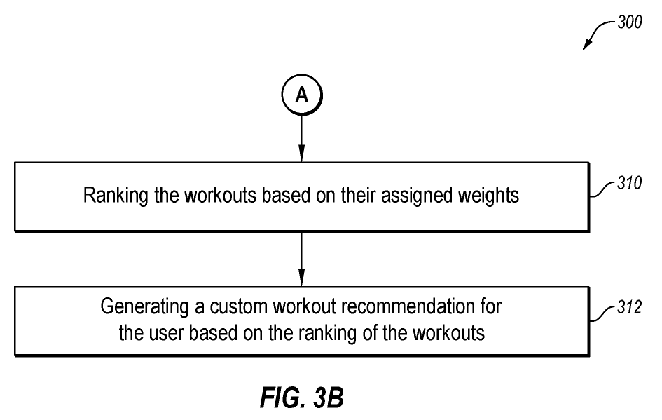

FIGS. 3A-3B are a diagram of an example method 300 for customizing workout recommendations. The method 300 may be performed, for example, by a software application being executed on the server 102, the laptop computer 104, the smartphone 106, the desktop computer 114, or the tablet 116, or some combination therefore, of FIG. 1.

The method 300 may include receiving, at 302, a target workout duration for a user, a target muscle group for the user, a target workout category goal of the user, a fitness level of the user, a workout equipment availability of the user, and a sex of the user.

The method 300 may include determining, at 304, a target calorie burn for the user.

The method 300 may include determining, at 306, the recentness of each of the workouts completed by the user and determining the recentness of each of the multiple target muscle groups being a focus of the workouts completed by the user. These determinations may include receiving physical movement data of the user from one or more electronic sensors configured to directly measure the physical movement of the user, analyzing the physical movement data, and determining whether each of the workouts was completed based on the analysis of the physical movement data.

The method 300 may include assigning, at 308, a weight to each of the workouts based on the received target workout duration, the received target muscle group for the user, the received target workout category goal of the user, the received fitness level of the user, the received workout equipment availability of the user, the received sex of the user, the determined target calorie burn for the user, the determined recentness of the workout being completed by the user, and the determined recentness of each of the multiple target muscle groups being a focus of the workouts completed by the user.

The method 300 may include ranking, at 310, the workouts based on their assigned weights.

The method 300 may include generating, at 312, a custom workout recommendation for the user based on the ranking of the workouts.

### INDUSTRIAL APPLICABILITY

In general, the methods for customizing workout recommendations disclosed above generate custom workout recommendations for users based on various data that is received or determined. Various modifications to the methods disclosed above will now be disclosed.

The software application disclosed herein that is configured to receive data, analyze data, make determinations with respect to data, and generate custom workout recommendations may be configured to be executed on one or more computing devices. For example, the computing devices may include, but are not limited to, an application or app that is executed on a smartphone, a smart watch, a smart panel of a smart home network, an exercise machine, a laptop computer, a tablet, or a desktop computer. Further, the software application may be distributed across two or more computing devices that communicate with each other over a wired or wireless network.

Further, the software application disclosed herein may be configured to execute according to one or more formulas. For example, the weight assigned to each workout by the software application disclosed herein may be calculated according to the following formula:

A*B*C*D*E*F*G*H*I*J=Workout Weight

where:

A=Recently completed workout weight

B=Recently recommended workout weight

C=Target muscle group frequency weight

D=Workout calorie burn weight

E=Target muscle group weight

F=Difficulty level weight

G=Target category goal weight

H=Equipment availability weight

I=Duration weight

J=Gender specificity weight

Using this formula, a weight=1 may be considered neutral, a weight >1 may be preferred, a 0<weight <1 may not preferred but allowed, and a weight=0 may not be allowed and never recommended. Since the weights for all different preference settings in this formula are multiplied together for each workout, if a workout has a total weight of 1.2, it is twice as likely to be recommended as a workout that has a weight of 0.6. For example, where a workout has not been completed (1) or recommended in the past 10 days (1), contains the target muscle group that was used three days ago (0.875), falls within the allowed calorie range (1), contains target muscle groups the user would like to focus on (1.5), is the difficulty level selected by the user (1), falls in the category the user has selected their goal (1), uses equipment the user has access to (1.2), is of the desired duration (1), and is gender neutral (1), the weight for the workout will be 1*1*0.875*1*1.5*1*1*1.2*1*1=1.575. With a weight of 1.575, this weight is far more likely to be recommended than average. Calculations of each of the individual weights A-J will now be described.

The following formula may be used to calculate the weight A, which affects how often a workout will be recommended again after it has been completed.

140.85\hat{ }(t−1))=A

This formula assumes that that t represents a number of days, with t=0 on the day the workout is completed, and this formula is only employed where t≤10. For example, if a user completed the workout four days ago, the weight A will be 1−(0.85\hat{ }(4−1))=0.385875. Once t=10, the weight A=1.

The following formula may be used to calculate the weight B, which affects how often a workout will be recommended again after it has been recommended but not completed.

1−(0.6\hat{ }t)=B

This formula assumes that t represents a number of days, t=0 on the day the workout is recommended but not logged, and this formula is only employed where t≤5. For example, if a user was recommended the workout three days ago but the workout was not completed, the weight B will be 1−(0.851\hat{ }3)=0.784. Once t=5, the weight B=1.

The following formula may be used to calculate the weight C, which affects how often a target muscle group will be the focus of a workout after a workout with the same target muscle group has been completed.

140.5\hat{ }t)=C

This formula assumes that t represents a number of days, t=0 on the day the workout is recommended but not logged, and this formula is only employed where t≤4. For example, if a user was completed a workout two days ago, a workout focusing on the same target muscle group with have a weight C=1−(0.5\hat{ }2)=0.75. Once t=4, the weight C=1.

The weight D may affect how often a workout will be recommended based on a target calorie burn. For example, a target calorie burn may fall within a range, and if a workout is more or less than the range, the weight D=0, otherwise the weight D=1. For example, if the target calorie burn is a range of 400 to 500 calories, workouts from 400 to 500 calories will have a weight D=1, and all other workouts will have a weight D=0. The weight D may be calculated by receiving a fitness level of the user, determining a target heart rate range for the user based on the received fitness level of the user, receiving a mass of the user, receiving the sex of the user, and determining the target calorie burn for the user based on the determined target heart rate range for the user, the received mass of the user, the received sex of the user, and the received target workout duration for the user. For example, the target calorie burn for males may be calculated according to the following formula:

((−55.0969+(0.6309*HR)±(0.1988*M)+(0.2017*A))*0.239005736)*T=Calorie Burn

Similarly, the target calorie burn for females may be calculated according to the following formula:

((−20.4022+(0.4472*HR)−(0.1263*M)±(0.074*A))*0.239005736)*T=Calorie Burn

where, in both formulas:

HR=Target heart rate range for the user

M=Mass

A=Age

T=Target workout duration

Heart rate ranges may be determined by fitness level, where a fitness level of a beginner is determined to have a heart rate range of 110-155, a fitness level of intermediate is determined to have a heart rate range of 120-165, and a fitness level of advanced is determined to have a heart rate range of 130-175. For example, the target calorie burn for a male with a workout time set to 25 minutes (or a workout time set to 20-30 minutes, which results in a midpoint of 25 minutes), and is 37 years old with a mass of 82 kg at an intermediate level would have a calorie burn target range (running from minimum to maximum) of:

Minimum: ((−55.0969+(0.6309*120)+(0.1988*82)+(0.2017*37))*0.239005736)*25=265

Maximum: ((−55.0969+(0.6309*165)+(0.1988*82)+(0.2017*37))*0.239005736)*25=434

In this example, workouts from 265 to 434 calories will have a weight D=1, and all other workouts will have a weight D=0.

The weight E may affect how often a workout will be recommend based on if the workout focuses on a target muscle group. For example, the weight E=1.5 if the workout focuses on a user's target muscle group, and the weight D=1 if the workout does not focus on the user's target muscle group.

The weight F may affect how often a workout will be recommended based on how closely the workout fits within the user's fitness level. For example, where fitness levels are divided into a fitness level of beginner, a fitness level of intermediate, and a fitness level of advanced, the weight F=1 if the workout falls within the user's fitness level, the weight F=0.6 if the workout falls only one level outside of the user's fitness level, and the weight F=0 if the workout falls two or more levels outside of the user's fitness level.

The weight G may affect how often a workout will be recommended based on how closely the workout fits within the user's target workout category goal. For example, where workout category goals are divided into (1) happy healthy, (2) lean and toned, (3) weight loss, and (4) strength and power, the weight G may have the following values. If happy and healthy is targeted, a workout with happy and healthy has a weight G=1, lean and tone or weight loss has a weight G=0.7, and strength and power has a weight G=0.3. If lean and tone is targeted, a workout with happy and healthy or weight loss has a weight G=0.7, lean and tone has a weight G=1.0, and strength and power has a weight G=0.3. If weight loss is targeted, a workout with happy and healthy or lean and tone has a weight G=0.7, happy and healthy has a weight G=1.0, and strength and power has a weight G=0.3. If strength and power is targeted, a workout with happy and healthy or lean and tone or weight loss has a weight G=0.3, and strength and power has a weight G=1.0.

The weight H may affect whether a workout will be recommended based on if the necessary workout equipment is available to the user. For example, the weight H=1.2 if the workout uses equipment available to the user, the weight H=1 if the workout requires no workout equipment, and the weight H=0 if the workout requires equipment to which the user does not have access.

The weight I may affect whether a workout will be recommended based on if the workout fits within the target workout duration of the user. For example, if the workout is shorter or longer than the target workout duration, the weight I=0.3, but if the workout is of the target workout duration, the weight I=1.

The weight J may affect whether a workout will be recommended based on whether the workout is targeted toward the sex of the user. For example, if the workout is targeted toward the sex of the user or is sex neutral, the weight J=1. If the workout is targeted toward the opposite sex from the user, the weight J=0.

It is understood that the various weights A-J employed in the Workout Weight formula above may be combined in a variety of ways including eliminating one or more of the weights A-J from the Workout Weight formula.

Further, the software application disclosed herein may include the use of a special-purpose or general-purpose computer, including various computer hardware or software. The software application may be implemented using non-transitory computer-readable media for carrying or having computer-executable instructions or data structures stored thereon. Such computer-readable media may be any available media that may be accessed by a general-purpose or special-purpose computer. By way of example, and not limitation, such computer-readable media may include non-transitory computer-readable storage media including RAM, ROM, EEPROM, CD-ROM or other optical disk storage, magnetic disk storage or other magnetic storage devices, or any other storage medium which may be used to carry or store one or more desired programs having program code in the form of computer-executable instructions or data structures and which may be accessed and executed by a general-purpose computer, special-purpose computer, or virtual computer such as a virtual machine. Combinations of the above may also be included within the scope of computer-readable media. Computer-executable instructions comprise, for example, instructions and data which, when executed by one or more processors, cause a general-purpose computer, special-purpose computer, or virtual computer such as a virtual machine to perform a certain method, function, or group of methods or functions.

The communication between computing devices disclosed herein may be accomplished over any wired or wireless communication network including, but not limited to, a Local Area Network (LAN), a Wide Area Network (WAN), a Wireless Application Protocol (WAP) network, a Bluetooth network, an ANT network, or an Internet Protocol (IP) network such as the Internet, or some combination thereof.

The receipt of data from a user disclosed herein in connection with various webpages of a website may additionally or alternatively be accomplished using other data gathering technologies including, but not limited to, receiving data from a user via data entry interfaces of an app on a smartphone or gathering data regarding a user by accessing databases that already store the desired data such as registration databases of an app server or a website server, or some combination thereof. Further, the receipt of data from a user disclosed herein in connection with various webpages of a website is example data only, and other types and specificity of data may additionally or alternatively be received from a user.

The electronic sensors disclosed herein that are configured to directly measure physical movement of the user may include both portable as well as stationary electronic sensors. Portable electronic sensors may include, but are not limited to, electronic sensors built into smart watches, fitness trackers, sport watches, head mounted displays, smart clothing, smart jewelry, vehicles, sports equipment, or implantables configured to be implanted in the human body, or some combination thereof. Stationary electronic sensors may include, but are not limited to, sensors built into exercise machines, furniture, beds or bedding (to measure physical movement while in bed and/or while asleep), flooring, walls, ceilings, doorways, or fixtures along paths and roadways, or some combination thereof. These sensors configured to measure physical movement of the user may include, but are not limited to, sensors that measure physical movement using infrared, microwave, ultrasonic, tomographic, GPS, accelerometer, gyroscope, odometer, tilt, speedometer, piezoelectric, or video technologies, or some combination thereof.

The use of one or more electronic sensors in the example methods disclosed herein may solve the problem of a subjective recommendation from a dietitian that is based on subjective information provided by a user. In particular, since a dietitian is a human being, the dietitian is inherently biased and any recommendations are necessarily subjective instead of objective. Further, there are severe limitations to what types of information, and accuracy of information, that a human user can gather and convey to the human dietitian. The use of one or more electronic sensors in the example methods disclosed herein may solve these problems by using highly sophisticated and specialized electronic sensors that are configured to objectively and directly measure physical movement of the user resulting in objective physical movement data and then sending that objective physical movement data to the objective software application disclosed herein instead of a subjective human dietitian. These electronic sensors may have specific tolerances and may enable a single computing device to measure multiple users in multiple remote locations. None of these capabilities are available to a human user absent these highly sophisticated and specialized electronic sensors. These highly sophisticated and specialized electronic sensors may therefore solve the problems with the prior art method by objectively and accurately measuring physical movement of the user instead of relying on subjective and biased observations of a user.

Further, the example methods disclosed herein are not directed to an abstract idea because they solve a technical problem using highly sophisticated and specialized electronic sensors. The data generated by these electronic sensors simply has no equivalent to pre-electronic sensor, manual paper-and-pencil data.

Also, the example methods disclosed herein may improve the technical field of automated workout recommendations. For example, the technical field of automated workout recommendations may be improved by the example methods disclosed herein at least because the prior art method did not enable the automatic measurement of the physical movement of a user and the automatic sending of physical movement data to a software application capable of customizing a workout recommendation based on an automatic analysis and determination of parameters from the received physical movement data.

1. A custom workout system comprising:
an exercise machine;
an electronic sensor built into the exercise machine and configured to directly measure physical movement of a user; and
an app configured to be executed on a smartphone or on a tablet, the app configured, when executed, to cause one or more processors of the smartphone or the tablet to perform a method for customizing workout recommendations, the method including:
determining a fitness level of the user;
determining a weight of the user;
determining a target calorie burn for the user based at least in part on the fitness level of the user and the weight of the user;
receiving physical movement data of the user from the electronic sensor;
analyzing the physical movement data to determine a physical movement parameter; and
generating a custom workout recommendation for the user to be performed in connection with the exercise machine based at least in part on the target calorie burn and the physical movement parameter.

2. The custom workout system of claim 1, wherein:
the custom workout system further comprises a server;
the app is further configured to execute distributed across the smartphone or the tablet and the server such that the app, when executed, causes the one or more processors of the smartphone or the tablet and one or more processors of the server to perform the method; and
the smartphone or the tablet and the server are configured to communicate with each other over the Internet.

3. The custom workout system of claim 1, wherein:
the method further includes determining a birthday of the user; and
the determining of the target calorie burn for the user is further based at least in part on the birthday of the user.

4. The custom workout system of claim 1, wherein:
the method further includes determining a gender of the user; and
the determining of the target calorie burn for the user is further based at least in part on the gender of the user.

5. The custom workout system of claim 1, wherein:
the method further includes determining a target workout category goal of the user; and
the generating of the custom workout recommendation for the user is further based at least in part on the target workout category goal.

6. The custom workout system of claim 5, wherein the target workout category goal includes a goal to improve one or more of health, muscle tone, weight loss, or muscle strength, or some combination thereof.

7. The custom workout system of claim 1, wherein:
the method further includes determining a target workout duration for the user; and
the generating of the custom workout recommendation for the user is further based at least in part on the target workout duration.

8. The custom workout system of claim 1, wherein the electronic sensor includes an electronic resistance sensor configured to track an amount of effort expended by the user on the exercise machine.

9. The custom workout system of claim 1, wherein the physical movement parameter includes a number of calories burned by the user.

10. The custom workout system of claim 1, wherein:
the custom workout system further comprises a wearable electronic sensor configured to be worn on an arm of the user and configured to track an amount of effort expended by the user on the exercise machine; and
the method further includes receiving second physical movement data of the user from the wearable electronic sensor; and
the analyzing of the physical movement data to determine the physical movement parameter further includes analyzing the second physical movement data.

11. A custom workout system comprising:
a server;
an exercise machine;
an electronic sensor built into the exercise machine and configured to directly measure physical movement of a user; and
an app configured to execute distributed across a smartphone or a tablet and the server, the smartphone or the tablet and the server configured to communicate with each other over the Internet, the app configured, when executed, to cause one or more processors of the smartphone or the tablet and the server to perform a method for customizing workout recommendations, the method including:
determining a fitness level of the user;
determining a weight of the user;
determining a target calorie burn for the user based at least in part on the fitness level of the user and the weight of the user;
receiving physical movement data of the user from the electronic sensor;
analyzing the physical movement data to determine a physical movement parameter; and
generating a custom workout recommendation for the user to be performed in connection with the exercise machine based at least in part on the target calorie burn and the physical movement parameter.

12. The custom workout system of claim 11, wherein the smartphone or the tablet and the server configured to communicate with each other over the Internet at least partially via a wireless Bluetooth network.

13. The custom workout system of claim 11, wherein:
the method further includes determining a birthday of the user; and
the determining of the target calorie burn for the user is further based at least in part on the birthday of the user.

14. The custom workout system of claim 11, wherein:
the method further includes determining a gender of the user; and
the determining of the target calorie burn for the user is further based at least in part on the gender of the user.

15. The custom workout system of claim 11, wherein:
the method further includes determining a target workout category goal of the user; and
the generating of the custom workout recommendation for the user is further based at least in part on the target workout category goal.

16. The custom workout system of claim 15, wherein the target workout category goal includes a goal to improve one or more of health, muscle tone, weight loss, or muscle strength, or some combination thereof.

17. The custom workout system of claim 11, wherein:
the method further includes determining a target workout duration for the user; and
the generating of the custom workout recommendation for the user is further based at least in part on the target workout duration.

18. The custom workout system of claim 11, wherein the electronic sensor includes an electronic resistance sensor configured to track an amount of effort expended by the user on the exercise machine.

19. The custom workout system of claim 11, wherein the physical movement parameter includes a number of calories burned by the user.

20. The custom workout system of claim 11, wherein:
the custom workout system further comprises a wearable electronic sensor configured to be worn on an arm of the user and configured to track an amount of effort expended by the user on the exercise machine; and
the method further includes receiving second physical movement data of the user from the wearable electronic sensor; and
the analyzing of the physical movement data to determine the physical movement parameter further includes analyzing the second physical movement data.
